(19) United States
(12) Patent Application Publication
(10) Pub. No.: US 2013/0158370 A1
(43) Pub. Date: Jun. 20, 2013

(54) SPONTANEOUS BREATHING TRIAL MANAGER

(71) Applicant: COVIDIEN LP, Mansfield, MA (US)

(72) Inventors: Peter Doyle, Vista, CA (US); Joseph Douglas Vandine, Newark, CA (US)

(73) Assignee: COVIDIEN LP, Mansfield, MA (US)

(21) Appl. No.: 13/769,726

(22) Filed: Feb. 18, 2013

Related U.S. Application Data
(63) Continuation of application No. 12/714,248, filed on Feb. 26, 2010.

(51) Int. Cl. A61B 5/0205; A61B 5/1455; A61B 5/091

(52) U.S. Cl. CPC A61B 5/0205; A61B 5/091; A61B 5/14551
USPC 600/301; 600/538; 600/484

(57) ABSTRACT

This disclosure describes systems and methods for conducting and terminating spontaneous breathing trials on patients receiving mechanical ventilation. The disclosure describes a novel spontaneous breathing trial manager for a medical ventilator with rapid initiation and continuous monitoring of a patient's tolerance of the spontaneous breathing trial and displaying of that tolerance as a function of time, which provides for bedside adjustment of the spontaneous breathing trial parameters and automatic termination of a spontaneous breathing trial based on a time interval expiration or poor patient tolerance of the SBT.

### INTRODUCTION

Medical ventilator systems have been long used to provide supplemental breathing support to patients. These ventilators typically comprise a source of pressurized gas which is fluidly connected to the patient through a conduit. In some systems, the patient after an extended period of ventilation is placed on spontaneous breathing trials (SBT). The spontaneous breathing trials help to determine whether the patient is ready to be weaned from ventilator support.

The SBT is often conducted at low levels of ventilator support for a varying and/or constant period of time. The patient typically remains on the ventilator during the SBT to allow for better monitoring (of their tolerance of the SBT). The bedside clinician sets the breathing mode, spontaneous breath type and all associated settings for the SBT (either under a protocol or on the order of a physician).

However, there may be occasions where the bedside clinician cannot remain at the bedside for the duration of the set SBT time interval or cannot immediately attend to the patient if the patient has exceeded limits of monitored variables indicating a failure of the trial. Accordingly, conducting a SBT inconveniently require the bedside clinician to remain with the patient or be available to the patient for the duration of the SBT interval.

### SUMMARY

This disclosure describes systems and methods for conducting and terminating spontaneous breathing trials on patients receiving mechanical ventilation. The disclosure describes a novel spontaneous breathing trial manager for a medical ventilator with rapid initiation and continuous monitoring of a patient's tolerance of the spontaneous breathing trial and displaying of that tolerance as a function of time, which provides for bedside adjustment of the spontaneous breathing trial parameters and automatic termination of a spontaneous breathing trial based on a time interval expiration or poor patient tolerance of the SBT.

This disclosure describes a method for managing a spontaneous breathing trial in a medical ventilator. The method includes performing the following steps:

a) initiating a spontaneous breathing trial for a patient being ventilated on a medical ventilator;

b) monitoring a plurality of sensors to obtain a plurality of sensor measurements during the spontaneous breathing trial;

c) determining whether at least one of the plurality of sensor measurements is outside of a desired range for a predetermined amount of time;

d) determining whether a RSBI calculation is outside of a desired range for a predetermined amount of time;

e) ending the spontaneous breathing trial based on at least one of a determination that at least one of the plurality of sensor measurements is outside of the desired range for the predetermined amount of time, the RSBI calculation is outside of the desired range for the predetermined amount of time, an inputted user command, and expiration of a spontaneous breathing trial period;

f) displaying at least one of the plurality of sensor measurements as a function of time for the spontaneous breathing trial; and

g) displaying a basis for the step of ending the spontaneous breathing trial for the patient being ventilated on the medical ventilator.

This disclosure also describes a medical ventilator system including: a processor; a gas regulator controlled by the processor, the gas regulator adapted to regulate a flow of gas from a gas supply to a patient via a patient circuit; a breath frequency sensor controlled by the processor, the breath frequency sensor is adapted to measure the breath frequency of the patient; a spontaneous tidal volume sensor controlled by the processor, the spontaneous tidal volume sensor is adapted to measure spontaneous tidal volume of the patient; a spontaneous exhalation volume sensor controlled by the processor, the spontaneous exhalation volume sensor is adapted to measure spontaneous exhalation volume of the patient; a SpO₂ sensor controlled by the processor, the SpO₂ sensor is adapted to measure blood oxygen saturation level of the patient; a heart rate sensor controlled by the processor, the heart rate sensor is adapted to measure heart rate of the patient; a spontaneous breathing trial manager in communication with the processor, the breath frequency sensor, the spontaneous tidal volume sensor, the spontaneous exhalation volume sensor, the SpO₂ sensor, and the heart rate sensor; a user interface in communication with the processor and the spontaneous breathing trial manager; and a display module controlled by the processor, the display module adapted to display RSBI and at least one of heart rate, blood oxygen saturation level, spontaneous tidal volume, and spontaneous exhalation volume of the patient as a function of time for a spontaneous breathing trial. The spontaneous breathing trial manager further includes a threshold monitor module and a ventilation module.

Yet, another aspect of the disclosure describes a pressure support system. The pressure support system includes: a processor; a pressure generating system adapted to generate a flow of breathing gas controlled by the processor; a ventilation system including a patient circuit controlled by the processor; a breath frequency sensor controlled by the processor, the breath frequency sensor is adapted to measure breath frequency of the patient; a spontaneous tidal volume sensor controlled by the processor, the spontaneous tidal volume sensor is adapted to measure spontaneous tidal volume of the patient; a spontaneous exhalation volume sensor controlled by the processor, the spontaneous exhalation volume sensor is adapted to measure spontaneous exhalation volume of the patient; a SpO₂ sensor controlled by the processor, the SpO₂ sensor is adapted to measure blood oxygen saturation level of the patient; a heart rate sensor controlled by the processor, the heart rate sensor is adapted to measure heart rate of the patient; a spontaneous breathing trial manager in communication with the processor, the breath frequency sensor, the spontaneous tidal volume sensor, the spontaneous exhalation volume sensor, the SpO₂ sensor, and the heart rate sensor; a user interface in communication with the processor and the spontaneous breathing trial manager; and a display module controlled by the processor, the display module adapted to display heart rate, RSBI, blood oxygen saturation level, spontaneous tidal volume, and spontaneous exhalation volume of the patient as a function of time for a spontaneous breathing trial. The spontaneous breathing trial manager further includes a threshold monitor module and a ventilation module.

These and various other features as well as advantages will be apparent from a reading of the following detailed description and a review of the associated drawings. Additional features are set forth in the description that follows and, in part, will be apparent from the description, or may be learned by practice of the described embodiments. The benefits and features will be realized and attained by the structure particularly pointed out in the written description and claims hereof as well as the appended drawings.

It is to be understood that both the foregoing general description and the following detailed description are exemplary and explanatory and are intended to provide further explanation of the claimed invention.

### BRIEF DESCRIPTION OF THE DRAWINGS

The following drawing figures, which form a part of this application, are illustrative of embodiments systems and methods described below and are not meant to limit the scope of the invention in any manner, which scope shall be based on the claims appended hereto.

FIG. 1 illustrates an embodiment of a ventilator connected to a human patient.

FIG. 2 illustrates an embodiment of an operatively coupled ventilator, spontaneous breathing trial manager, and display.

FIG. 3 illustrates an embodiment of a spontaneous breathing trial method for a medical ventilator.

FIG. 4 illustrates an embodiment of a display screen shot for a spontaneous breathing trial listing the ventilator parameters of a spontaneous breathing trial and user interface commands.

FIG. 5 illustrates an embodiment of a display screen shot for a spontaneous breathing trial on a medical ventilator graphing key patient variables verses time for the spontaneous breathing trial.

FIG. 6 illustrates an embodiment of a display screen shot for a spontaneous breathing trial on a medical ventilator graphing key patient variables verses time for the spontaneous breathing trial and the cause for ending the spontaneous breathing trial.

### DETAILED DESCRIPTION

Although the techniques introduced above and discussed in detail below may be implemented for a variety of medical devices, the present disclosure will discuss the implementation of these techniques in the context of a medical ventilator for use in providing ventilation support to a human patient. The reader will understand that the technology described in the context of a medical ventilator for human patients could be adapted for use with other systems such as ventilators for non-human patients and general gas transport systems in which periodic gas mixture changes may be required. As utilized herein a “gas mixture” includes at least one of a breathing gas and a mixture of breathing gases.

Medical ventilators are used to provide a breathing gas to a patient who may otherwise be unable to breathe sufficiently. In modern medical facilities, pressurized air and oxygen sources are often available from wall outlets. Accordingly, ventilators may provide pressure regulating valves (or regulators) connected to centralized sources of pressurized air and pressurized oxygen. The regulating valves function to regulate flow so that respiratory gas having a desired concentration of oxygen and other gases is supplied to the patient at desired pressures and rates. Ventilators capable of operating independently of external sources of pressurized air are also available.

While operating a ventilator, it can be desirable to provide spontaneous breathing trials (SBTs) that do not require the clinician to be present at the end of the set SBT time interval or available in case the patient exceeds a key variable threshold during the SBT.

Accordingly, a SBT manager for rapid initiation of SBTs (using institution-configured setting with flexibility for bedside adjustment, including desired duration) that monitors key variables to determine the patient's tolerance to the SBTs for a medical ventilator is desirable. The SBT manager automatically returns a patient to the previous (prior to SBT) ventilator settings in the event the preset time has elapsed or the patient has exceeded a clinician-set monitored variable thresholds. Further, the SBT manager records the trend of the patient's progress during the SBT and any causes for resumption of the previous setting, if this occurred for clinician review.

The SBT manager provides for several advantages. In one embodiment, the SBT manager improves the ease of use of the ventilator and a SBT. In a further embodiment, the SBT manager decreases the amount of time a clinician must monitor a patient during a SBT than previously utilized SBT ventilator systems. In another embodiment, the SBT manager decreases the amount of time necessary to program and/or initiate a SBT by a clinician than previously utilized SBT ventilator systems. In an additional embodiment, the SBT manager provides for better ventilator adherence to protocols than previously utilized SBT ventilator systems.

Those skilled in the art will recognize that the methods and systems of the present disclosure may be implemented in many manners and as such are not to be limited by the foregoing exemplary embodiments and examples. In other words, functional elements being performed by a single or multiple components, in various combinations of hardware and software or firmware, and individual functions, can be distributed among software applications, which may be distributed among one or multiple processors. In this regard, any number of the features of the different embodiments described herein may be combined into single or multiple embodiments, and alternate embodiments having fewer than or more than all of the features herein described are possible. Functionality may also be, in whole or in part, distributed among multiple components, in manners now known or to become known. Thus, myriad software/hardware/firmware combinations are possible in achieving the functions, features, interfaces and preferences described herein. Moreover, the scope of the present disclosure covers conventionally known manners for carrying out the described features and functions and interfaces, and those variations and modifications that may be made to the hardware or software or firmware components described herein as would be understood by those skilled in the art now and hereafter.

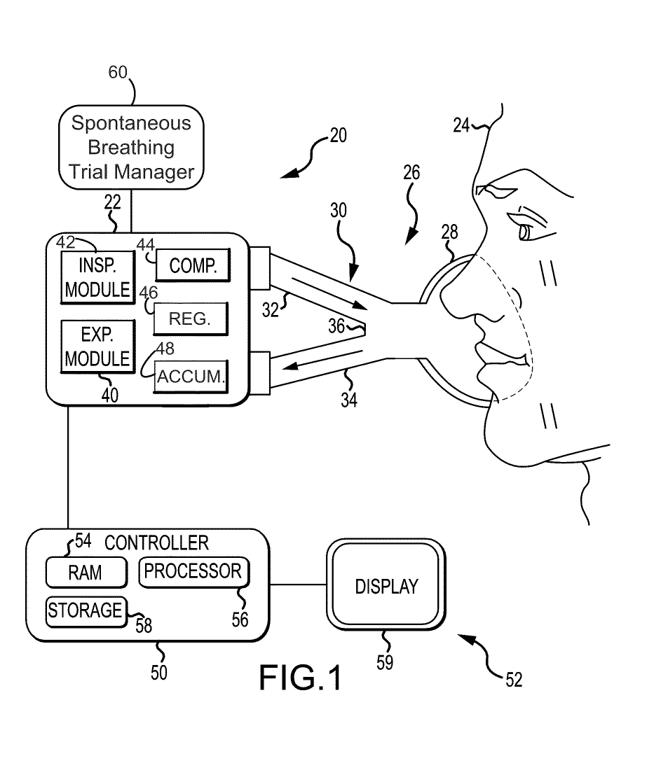

FIG. 1 illustrates an embodiment of a ventilator 20 connected to a human patient 24. Ventilator 20 includes a pneumatic system 22 (also referred to as a pressure generating system 22) for circulating breathing gases to and from patient 24 via the ventilation tubing system 26, which couples the patient 24 to the pneumatic system 22 via physical patient interface 28 and ventilator circuit 30. Ventilator circuit 30 could be a two-limb or one-limb circuit for carrying gas mixture to and from the patient 24. In a two-limb embodiment as shown, a wye fitting 36 may be provided to couple the patient interface 28 to the inspiratory limb 32 and the expiratory limb 34 of the circuit 30.

The present systems and methods have proved particularly advantageous in invasive settings, such as with endotracheal tubes. However, condensation and mucus buildup do occur in a variety of settings, and the present description contemplates that the patient interface 28 may be invasive or non-invasive, and of any configuration suitable for communicating a flow of breathing gas from the patient circuit 30 to an airway of the patient 24. Examples of suitable patient interface 28 devices include a nasal mask, nasal/oral mask (which is shown in FIG. 1), nasal prong, full-face mask, tracheal tube, endotracheal tube, nasal pillow, etc.

Pneumatic system 22 may be configured in a variety of ways. In the present example, system 22 includes an expiratory module 40 coupled with an expiratory limb 34 and an inspiratory module 42 coupled with an inspiratory limb 32. Further, the gas concentrations can be mixed and/or stored in a chamber of a gas accumulator 44 at a high pressure to improve the control of delivery of respiratory gas to the ventilator circuit 30. The inspiratory module 42 is coupled to the gas regulator 46 and accumulator 44 to control the gas mixture of pressurized breathing gas for ventilatory support via inspiratory limb 32.

The pneumatic system 22 may include a variety of other components, including other sources for pressurized air and/or oxygen, mixing modules, valves, sensors, tubing, filters, etc. In one embodiment, the pneumatic system 22 includes at least one of a breathing frequency sensor, a spontaneous tidal volume (V_{t spont}) sensor, a spontaneous exhalation volume (V_{e spont}) sensor, a carbon dioxide elimination sensor, a SpO₂ sensor, and a heart rate sensor. In another embodiment, the pneumatic system 22 includes a breath frequency sensor and at least one of a spontaneous tidal volume (V_{t spont}) sensor, a spontaneous exhalation volume (V_{e spont}) sensor, a carbon dioxide elimination sensor, a blood oxygen saturation level (SpO₂) sensor, and a heart rate sensor.

As shown, ventilator 20 further includes a spontaneous breathing trial manager 60 operatively coupled to the controller 50 and the pneumatic system 22. In one embodiment, the spontaneous breathing trial manager 60 is a separate independent component from ventilator 20. In an alternative embodiment, the spontaneous breathing trial manager 60 is incorporated in pneumatic system 22.

The spontaneous breathing trial manager 60 initiates a spontaneous breathing trial based on preset configurations, inputted command, or a selected mode. The SBT manager 60 provides for rapid initiation of SBTs (using institution- or factory-configured settings with flexibility for bedside adjustment, including desired duration) that monitors key variables to determine the patient's tolerance of the SBTs. In one embodiment, the key variables include at least one of a ratio of respiratory frequency in respirations per minute to tidal volume in liters (f/Vₜ) or as otherwise known as a rapid shallow breathing index (RSBI), spontaneous tidal volume (V_{t spont}), spontaneous exhalation volume (V_{e spont}), carbon dioxide elimination levels, blood oxygen saturation level (SpO₂), heart rate and the patient's breathing work estimate. The RSBI is calculated by utilizing an algorithm run by the processor. In another embodiment, the key variables include the ratio of respiratory frequency in respirations per minute to tidal volume in liters (f/Vₜ) or rapid shallow breathing index (RSBI) and at least one of spontaneous tidal volume (V_{t spont}), spontaneous exhalation volume (V_{e spont}), carbon dioxide elimination levels, blood oxygen saturation level (SpO₂), heart rate and the patient's breathing work estimate. The patient's breathing work estimate is determined when the ventilator is in a proportional assist ventilation mode or option. The SBT manager 60 automatically returns a patient 24 to the previous (prior to SBT) ventilator settings in the event the preset time has elapsed or the patient 24 has exceeded the clinician-set monitored variable thresholds. Further, the SBT manager 60 records the trend of the patient's progress during the SBT and any causes for resumption of the previous setting, if this occurred for clinician review. In one embodiment, the SBT manager 60 sends the patient's progress during the SBT to the display 59 for user viewing.

In the illustrated embodiment, ventilator 20 includes a display 59. The SBT manager 60 is operatively coupled to the ventilator display 59. In an alternative embodiment, the SBT manager 60 is operatively coupled to a separate display 59 component that is independent of the SBT manger 60 and the ventilator 20. In another embodiment, the SBT manager 60 includes a display 59.

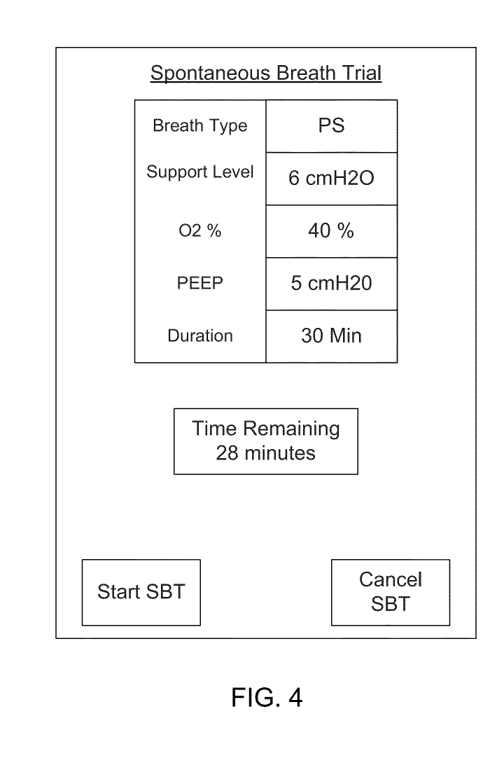

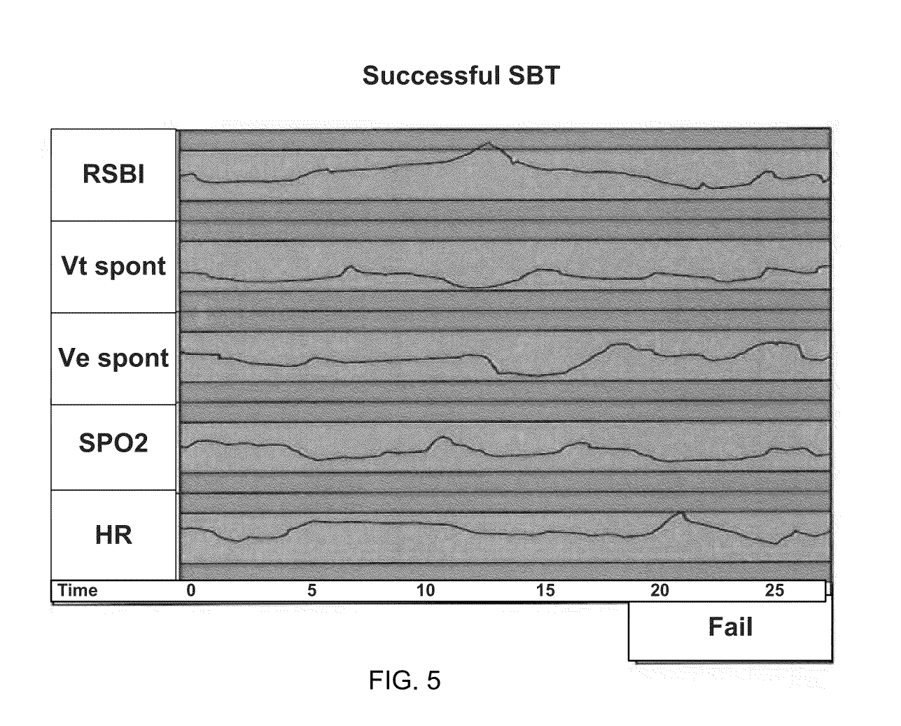

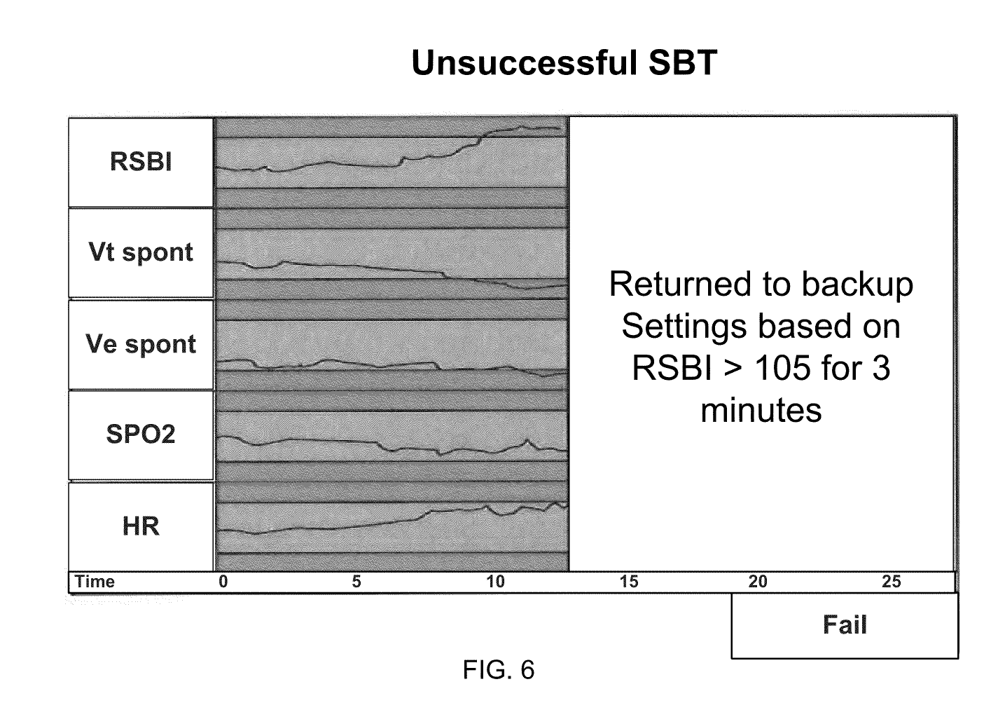

The display 59 can display any type of ventilation, patient, or SBT manager information, such as sensor readings, parameters, commands, alarms, warnings, and smart prompts (i.e., ventilator determined operator suggestions). In one embodiment, the display 59 lists the breath type utilized by ventilator 20, the pressure support level, the percentage of oxygen in the gas mixture, the positive end-expiratory pressure (PEEP), the predetermined amount of time for the SBT trial, and the amount of time remaining of the SBT period, as illustrated in FIG. 4. In another embodiment, the display 59 may show the trend of the patient's progress as a function of time during the SBT, as illustrated in FIGS. 5 and 6. In one embodiment, the display illustrates at least one of a RSBI calculation, a spontaneous tidal volume measurement (V_{t spont}), a spontaneous exhalation volume (V_{e spont}) measurement, a carbon dioxide elimination measurement, a SpO₂ measurement, patient's breathing work estimate, and a heart rate measurement as a function of time. In another embodiment, the display illustrates the RSBI calculation and at least one of a spontaneous tidal volume measurement (V_{t spont}), a spontaneous exhalation volume (V_{e spont}) measurement, a carbon dioxide elimination measurement, a SpO₂ measurement, patient's breathing work estimate, and a heart rate measurement as a function of time. Further, in the depicted example, the display 59 includes an operator interface 52 that is touch-sensitive, enabling the display 59 to serve both as an input user interface and an output device.

Controller 50 is operatively coupled with pneumatic system 22, SBT manager 60 signal measurement and acquisition systems, and an operator interface 52 may be provided to enable an operator to interact with the ventilator 20 (e.g., change ventilator settings, select operational modes, view monitored parameters, etc.). Controller 50 may include memory 54, one or more processors 56, storage 58, and/or other components of the type commonly found in command and control computing devices.

The memory 54 is non-transitory computer-readable storage media that stores software that is executed by the processor 56 and which controls the operation of the ventilator 20. In an embodiment, the memory 54 comprises one or more solid-state storage devices such as flash memory chips. In an alternative embodiment, the memory 54 may be mass storage connected to the processor 56 through a mass storage controller (not shown) and a communications bus (not shown). Although the description of non-transitory computer-readable media contained herein refers to a solid-state storage, it should be appreciated by those skilled in the art that non-transitory computer-readable storage media can be any available media that can be accessed by the processor 56. Non-transitory computer-readable storage media includes volatile and non-volatile, removable and non-removable media implemented in any method or technology for storage of information such as non-transitory computer-readable instructions, data structures, program modules or other data. Non-transitory computer-readable storage media includes, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the processor 56.

In another embodiment, the program may be run in working memory or working volatile memory. The working volatile memory must be reloaded at each initiation and may consist of RAM, DRAM, SDRAM, and selected mainly for speed of access and execution.

The controller 50 issues commands to pneumatic system 22 in order to control the breathing assistance provided to the patient 24 by the ventilator 20. The commands may be based on inputs received from patient 24, pneumatic system 22 and sensors, operator interface 52, SBT manager 60, and/or other components of the ventilator 20.

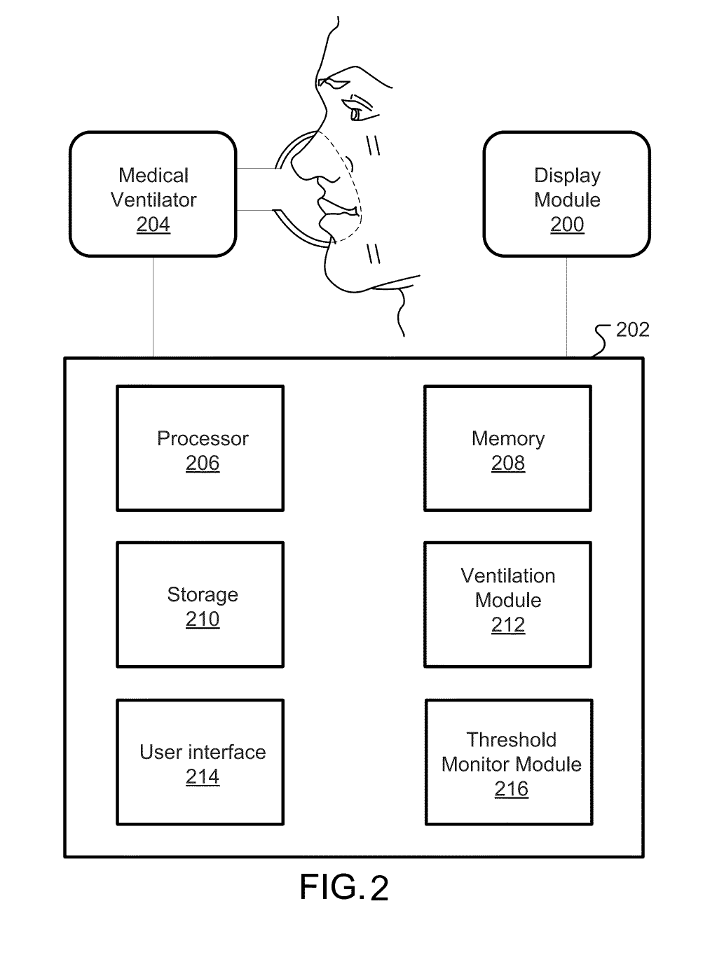

FIG. 2 illustrates an embodiment of a spontaneous breathing trial manager 202 (SBT manager 202) operatively coupled with a medical ventilator 204 and a display module 200. SBT manager 202 may include memory 208, one or more processors 206, storage 210, and/or other components of the type commonly found in command and control computing devices.

The memory 208 is non-transitory computer-readable storage media that stores software that is executed by the processor 206 to determine commands to send to the ventilator 204 for controlling the ventilator settings. In an embodiment, the memory 208 comprises one or more solid-state storage devices such as flash memory chips. In an alternative embodiment, the memory 208 may be mass storage connected to the processor 206 through a mass storage controller (not shown) and a communications bus (not shown). Although the description of non-transitory computer-readable media contained herein refers to a solid-state storage, it should be appreciated by those skilled in the art that non-transitory computer-readable storage media can be any available media that can be accessed by the processor 206. Non-transitory computer-readable storage media includes volatile and non-volatile, removable and non-removable media implemented in any method or technology for storage of information such as non-transitory computer-readable instructions, data structures, program modules or other data. Non-transitory computer-readable storage media includes, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the processor 206.

In an embodiment, the SBT manager 202 sends commands to the ventilator 204 or to the pneumatic system of the ventilator 204 in order to control ventilator settings. In another embodiment, a SBT manager 202 provides for quick set-up and rapid initiation of SBTs (using institution-configured setting with flexibility for bedside adjustment, including the predetermined amount of time for the SBT) that monitors key variables to determine the patient's tolerance to the SBTs for a medical ventilator 204.

In one embodiment, the SBT manager 202 monitors key variables by receiving sensor measurements. In another embodiment, the SBT manager 202 monitors key variable by communicating with the processor. The processor may monitor the key variables by receiving sensor measurements. In one embodiment, the medical ventilator 204 includes at least one of a breath frequency sensor, a spontaneous tidal volume (V_{t spont}) sensor, a spontaneous exhalation volume (V_{e spont}) sensor, a carbon dioxide elimination sensor, a blood oxygen saturation level (SpO₂) sensor, patient's breathing work estimate, and heart rate sensor. In another embodiment, the medical ventilator 204 includes a rapid shallow breathing index (RSBI) monitor and at least one of a breath frequency sensor, a spontaneous tidal volume (V_{t spont}) sensor, a spontaneous exhalation volume (V_{e spont}) sensor, a carbon dioxide elimination sensor, a blood oxygen saturation level (SpO₂) sensor, and heart rate sensor. In another embodiment, the medical ventilator 204 includes a rapid shallow breathing index (RSBI) monitor, a breath frequency sensor, a spontaneous tidal volume (V_{t spont}) sensor, a spontaneous exhalation volume (V_{e spont}) sensor, a blood oxygen saturation level (SpO₂) sensor, and heart rate sensor.

Any ventilator parameter suitable for affecting a SBT may be adjusted by a user through the SBT manger 202 during a SBT. In one embodiment, the support level, the oxygen percentage of the gas mixture, PEEP, trial time period, and/or breath type of the ventilator can be adjusted by a user through the SBT manager 202.

In one embodiment, the SBT manager 202 automatically returns a patient to the previous (prior to SBT) ventilator settings in the event the predetermined time has elapsed or the patient has exceeded the clinician-set monitored variable thresholds. Accordingly, the SBT manager 202 decreases the amount of time a clinician must monitor a patient during a SBT compared to previously utilized SBT ventilator systems. Further, the SBT manager 202 provides for better ventilator adherence to protocols than previously utilized SBT ventilator systems.

Additionally, the SBT manager 202 records the trend of the patient's progress during the SBT and any causes for resumption of the previous setting, if this occurred for clinician review.

As shown, the SBT manager 202 is operatively coupled to a separate and independent display module 200. In an alternative embodiment, the display module 200 is incorporated in the ventilator or SBT manager 202. The display module 200 is suitable for displaying ventilator information, patient information, and/or SBT information. In one embodiment, the display lists the breath type, support level, oxygen percentage of the gas mixture, PEEP, time period, and/or the time remaining of the SBT period, as illustrated in FIG. 4.

In one embodiment, the display module 200 is touch-sensitive, enabling the display to serve both as an input user interface and an output device. The user interface 214 allows a user to input commands, patient information, ventilator parameters, and SBT parameters. In one embodiment, the user interface 214 allows a user to start a SBT or cancel an already occurring SBT, as illustrated in FIG. 4. In another embodiment, the user interface 214 in the interactive display allows a user to change the predetermined amount of time for the SBT during a SBT period. Accordingly, the SBT manager 202 improves the ease of use of the ventilator and a SBT compared to previously utilized SBT systems.

In a further embodiment, the display module 200 illustrates the trend of the patient's progress during the SBT and any causes for resumption of the previous setting, if this occurred for clinician review. In one embodiment, the display graphically depicts a patient's progress during the SBT as a function of time for the SBT period. The patient's progress may be determined by monitoring different sensor measurements. In one embodiment, the patient's progress during the SBT is depicted by showing the rapid shallow breathing index (RSBI), respiration rate, spontaneous tidal volume (V_{t spont}) spontaneous exhalation volume (V_{e spont}), blood oxygen saturation level (SpO₂), and heart rate as a function of time, as illustrated in FIGS. 5 and 6. In another embodiment, the display illustrates at least one of a ratio of respiratory frequency in respirations per minute to tidal volume in liters (f/Vₜ), a carbon dioxide elimination level, a rapid shallow breathing index (RSBI), a respiration rate, a breathing work estimate, a spontaneous tidal volume (V_{t spont}), a spontaneous exhalation volume (V_{e spont}), a blood oxygen saturation level (SpO₂), and a heart rate as a function of time. In another embodiment, the display illustrates at least one of a ratio of respiratory frequency in respirations per minute to tidal volume in liters (f/Vₜ) or a RSBI and at least one of a carbon dioxide elimination level, a rapid shallow breathing index (RSBI), a respiration rate, a spontaneous tidal volume (V_{t spont}), a heart exhalation volume (V_{e spont}), a blood oxygen saturation level (SpO₂), and a heart rate as a function of time.

As illustrated in FIG. 6, the reason for a failed SBT trial is shown on the display. In this embodiment, the RSBI exceeded the desired range for three minutes and the spontaneous tidal volume is below the desired range for a period of time; therefore, the SBT manager 202 terminated the SBT. In another embodiment, the SBT manager ended the SBT because RSBI and at least one of a carbon dioxide elimination measurement, a respiration rate measurement, a spontaneous tidal volume (V_{t spont}) measurement, a breathing work estimate, a spontaneous exhalation volume (V_{e spont}) measurement, a blood oxygen saturation level (SpO₂) measurement, and a heart rate measurement is outside of a desired range for a period of time. In a further embodiment, the SBT manager ended the SBT because RSBI is outside the desired range for three minutes and at least one of a carbon dioxide elimination measurement, a respiration rate measurement, a spontaneous tidal volume (V_{t spont}) measurement, a spontaneous exhalation volume (V_{e spont}) measurement, a blood oxygen saturation level (SpO₂) measurement, and a heart rate measurement is outside of a desired range for 5 seconds. In another embodiment, at least one of a ratio of respiratory frequency in respirations per minute to tidal volume in liters (f/Vₜ), a carbon dioxide elimination level, a rapid shallow breathing index (RSBI), a respiration rate, a spontaneous tidal volume (V_{t spont}), a spontaneous exhalation volume (V_{e spont}), a blood oxygen saturation level (SpO₂), and a heart rate are outside of their desired threshold for a period of time. In another embodiment, at least two of a carbon dioxide elimination measurement, a respiration rate measurement, a spontaneous tidal volume (V_{t spont}) measurement, a spontaneous exhalation volume (V_{e spont}) measurement, a blood oxygen saturation level (SpO₂) measurement, and a heart rate measurement are outside of their desired range for period time, such as three minutes. These embodiments are not limiting. Any suitable combination of exceeded parameters for any suitable period of time can be utilized to terminate a SBT. Further, any reason for termination of a SBT may be shown on the display monitor.

In the embodiment shown, the SBT manager 202 further includes a ventilation module 212, a user interface 214, and a threshold monitor module 216. The threshold monitor module 216 utilizes ventilator and patient information to monitor the patient's tolerance of the SBTs for the medical ventilator 204. The threshold monitor module 216 determines if key variables are within a desired range or beyond a desired threshold or range. The key variable may be monitored through sensor measurements. In one embodiment, the threshold monitor module 216 determines if key variables are within a desired range or beyond a desired threshold for a predetermined amount of time. The key variables are any suitable ventilator or patient information that is an indicator of the patient's tolerance to the SBT. In one embodiment, the key variables include the ratio of respiratory frequency in respirations per minute to tidal volume in liters (f/Vₜ), rapid shallow breathing index (RSBI), spontaneous tidal volume (V_{t spont}) spontaneous exhalation volume (V_{e spont}), blood oxygen saturation level (SpO₂), carbon dioxide elimination levels (V_{CO2}), and/or heart rate. Each key variable has a desired range for the patient during a SBT. One embodiment of desired thresholds for a patient during a SBT is illustrated in Table. 1 below:

TABLE 1
Example Thresholds for Key Variables During a SBT

| Key Variable | Threshold |
|---|---|
| Respiration Rate | >35 breaths per min for a period of 5 minutes to |
| | <8 breaths per minute for a period of greater than 30 |
| | seconds |
| SpO₂ | <90% O₂ for a period of 3 minutes |
| Heart Rate | >130 beats per minute or |
| | a heart beat changes of 20% |
| RSBI | >105 |
| V_{CO2} | <150 mL/min or |
| | <85% of V_{CO2} prior to start of SBT or |
| | an increase of V_{CO2} >25% over the V_{CO2} prior to the |
| | start of the SBT |
| V_{t spont} | <3.5 mL/kg of preferred body weight |
| V_{e spont} | <60 mL/kg of preferred body weight per minute |
| Work Estimate | >1.2 Joules/L |

The thresholds listed in Table 1 above are exemplary only and are not limiting.

The threshold monitor module 216 notifies the ventilator module 212 as soon as a key variable exceeds a threshold value or falls outside of a desired range. Further, in one embodiment, the threshold monitor module 216 times the SBT period. In this embodiment, the threshold monitor module 216 notifies the ventilator module 212 as soon as the SBT period ends. Additionally, the threshold monitor module 216 may store this information in storage 210 or send it for display on the display module 200.

The ventilation module 212 may send commands to the ventilator 204. In one embodiment, the ventilation module 212 utilizes ventilator information, patient information, inputted parameters and commands, and/or threshold monitoring module information to determine the proper ventilator commands In one embodiment, the ventilator module 212 commands the medical ventilator 204 to initiate a SBT, return to previous ventilator settings, alter the predetermined amount of time for a SBT, end a SBT, change a breath type of a SBT, alter the parameters of a SBT, and/or alter ventilator settings. For example, if the predetermined amount of time for the SBT expires, the ventilation module 212 may command the medical ventilator 204 to return to the ventilator settings utilized before the initiation of the SBT. In another example, the ventilation module 212 may command the ventilator to change a SBT ventilator setting based on new user inputted information.

The user interface 214 of the SBT manger 202 allows a user to adjust SBT parameters, ventilator parameter, and patient information suitable for affecting a SBT during a SBT. In one embodiment, the support level, the oxygen percentage of the gas mixture, PEEP, trial period, and/or breath type of the ventilator can be adjusted by a user through the SBT manager 202. In an alternative embodiment, the user interface 214 is a touch sensitive display. In the embodiment shown, the user interface 214 is a data entry station, such a keyboard. In one embodiment, the user interface 214 may generate smart prompts or ventilator setting recommendations or SBT protocols for a SBT based on patient and ventilator information, which are displayed by the display module 200. In another embodiment, the user interface 214 may recommend the initiation of a SBT based on patient and ventilator information, which is displayed through the display module 200. The user interface 214 sends all user commands and information to the ventilation module 212. In one embodiment, displayed user interface information can provide for quick set-up and rapid activation of a SBT for an operator. Accordingly, the SBT manager 202 decreases the amount time necessary to program and/or initiate a SBT by a clinician compared to previously utilized SBT systems.

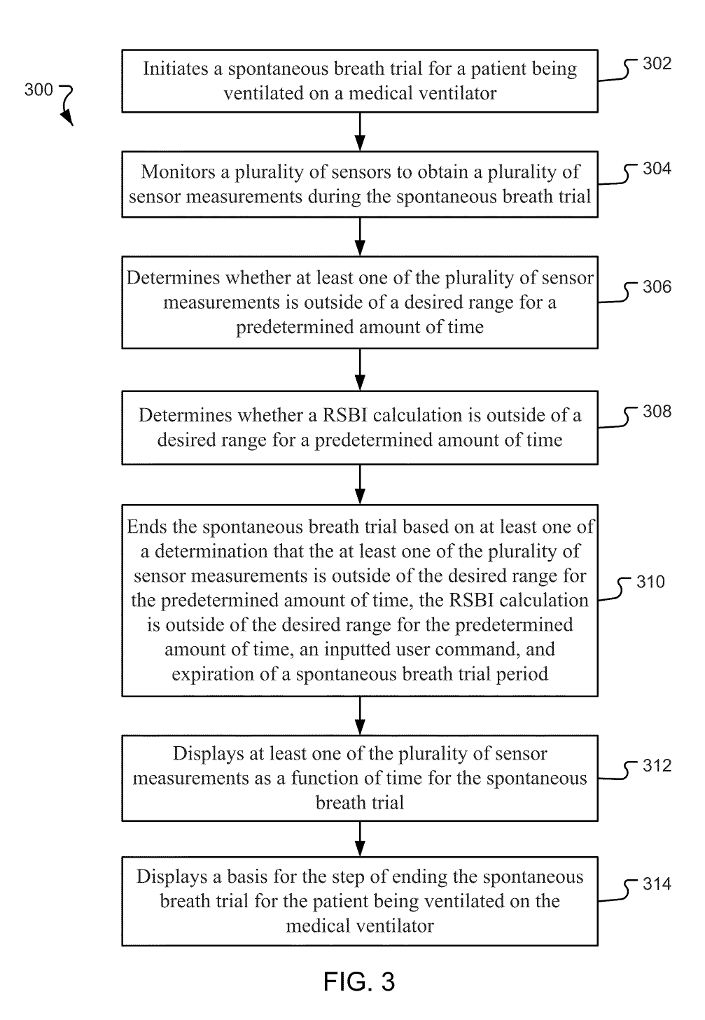

FIG. 3 represents an embodiment of a method for managing a spontaneous breathing trial in a medical ventilator 300. In one embodiment, method 300 modifies the spontaneous breathing trial based on at least one of user inputted parameters and user inputted commands during operation of the spontaneous breathing trial. In another embodiment, method 300 recommends spontaneous breathing trial ventilator parameters to an operator for the patient based on at least of past and present ventilation information and past and present patient information. In this embodiment, the operator may choose to ignore recommended parameters, partially utilize recommended parameters, or fully utilize recommended parameters.

As illustrated, method 300 initiates a spontaneous breathing trial for a patient being ventilated on a medical ventilator 302. In one embodiment, method 300 initiates the breathing trial based on user command In another embodiment, method 300 initiates the breathing trial based on preconfigured conditions. In a further embodiment, method 300 initiates the breathing trial based on preset conditions entered or selected by the operator. In an additional embodiment, method 300 initiates the breathing trial based on an inputted user parameter. In one embodiment, the predetermined amount of time for the SBT is 30 minutes. In another embodiment, the predetermined amount of time for the SBT is 45 minutes. The previous embodiments are not meant to be limiting. Any suitable predetermined amount of time for a SBT may be utilized by method 300.

Further, method 300 monitors a plurality of sensors to obtain a plurality of sensor measurements during the spontaneous breathing trial 304. In one embodiment, method 300 monitors at least one of a breath frequency sensor, a spontaneous tidal volume (V_{t spont}) sensor, a spontaneous exhalation volume (V_{e spont}) sensor, a carbon dioxide elimination sensor, a SpO₂ sensor, and a heart rate sensor. In another embodiment, method 300 obtains at least one of a breath frequency, an RSBI, a spontaneous tidal volume (V_{t spont}), a spontaneous exhalation volume (V_{e spont}), a carbon dioxide elimination, a SpO₂, and a heart rate measurement. In another embodiment, the sensor measurements includes breath frequency and at least one of respiration rate, carbon dioxide elimination levels, spontaneous tidal volume, spontaneous exhalation volume, blood oxygen saturation level, and heart rate. In a further embodiment, the sensor measurements are breath frequency, spontaneous tidal volume, spontaneous exhalation volume, blood oxygen saturation level, and heart rate. The plurality of sensors may be located within the ventilator and/or may be external to the ventilator.

Next, method 300 determines whether at least one of the plurality of sensor measurements is outside of a desired range for a predetermined amount of time 306. Further, method 300 determines whether a rapid shallow breathing index (RSBI) calculation is outside of a desired range for a predetermined amount of time 308. The RSBI is calculated by utilizing an algorithm run by the processor.

The predetermined amount of time may be different for different measurements. Further, the predetermined amount of time may change when more than one measurement is outside of a desired range at one time. In one embodiment, the predetermined amount of time is 3 minutes. In another embodiment, the predetermined amount of time is 30 seconds. For example, in one embodiment, the RSBI calculation must exceed a desired range for 3 minutes unless another measurement is exceeded for time period of 30 seconds causing the desired RSBI violation time to shorten.

Method 300 ends the spontaneous breathing trial based on at least one of a determination that at least one of the plurality of sensor measurements is outside of the desired range for the predetermined amount of time, the RSBI calculation is outside of the desired range for the predetermined amount of time, an inputted user command, and expiration of a spontaneous breathing trial period 310. In one embodiment, method 300 ends the spontaneous breathing trial based on at least one of the RSBI calculation, a breath frequency sensor measurement, a respiration rate measurement, a carbon dioxide elimination measurement, a spontaneous tidal volume measurement, a spontaneous exhalation volume measurement, a blood oxygen saturation measurement, and a heart rate measurement being outside the desired range for three minutes. In one embodiment, method 300 ends the spontaneous breathing trial based on the RSBI calculation and at least one of a respiration rate measurement, a carbon dioxide elimination measurement, a spontaneous tidal volume measurement, a breath frequency measurement, a spontaneous exhalation volume measurement, a blood oxygen saturation measurement, and a heart rate measurement being outside the desired range for three minutes. In another embodiment, method 300 ends the spontaneous breathing trial based on the RSBI calculation being outside the desired range for three minutes and at least one of a respiration rate measurement, a carbon dioxide elimination measurement, a breath frequency measurement, a spontaneous tidal volume measurement, a spontaneous exhalation volume measurement, a blood oxygen saturation level measurement, and a heart rate measurement being outside the desired range for about 5 seconds. In a further embodiment, method 300 ends the spontaneous breathing trial based on at least two of a respiration rate measurement, a carbon dioxide elimination measurement, a spontaneous tidal volume measurement, a spontaneous exhalation volume measurement, a blood oxygen saturation measurement, and a heart rate measurement being outside the desired range for one minute.

As shown, method 300 displays at least one of the plurality of sensor measurements as a function of time for the spontaneous breathing trial 312. This display allows an operator to see trends in measurements for the SBT period. In one embodiment, method 300 displays at least one of spontaneous tidal volume, breath frequency, spontaneous exhalation volume, blood oxygen saturation level, carbon dioxide elimination levels, and heart rate as a function of time for the spontaneous breathing trial. In another embodiment, method 300 displays the RSBI calculation as a function of time for the spontaneous breathing trial. In this embodiment, method 300 displays the RSBI calculation as function time and at least one of spontaneous tidal volume, spontaneous exhalation volume, blood oxygen saturation level, breath frequency, carbon dioxide elimination levels, and heart rate as a function of time for the spontaneous breathing trial. In a further embodiment, method 300 displays the RSBI calculation, spontaneous tidal volume, spontaneous exhalation volume, blood oxygen saturation level, and heart rate as a function of time for the spontaneous breathing trial. In yet another embodiment, method 300 displays at least two of spontaneous tidal volume, spontaneous exhalation volume, blood oxygen saturation level, carbon dioxide elimination levels, breath frequency, and heart rate as a function of time for the spontaneous breathing trial.

Further, method 300 displays a basis for the step of ending the spontaneous breathing trial for the patient being ventilated on the medical ventilator 314. In one embodiment, method 300 displays that the predetermined amount of time for the SBT expired as the basis for ending the spontaneous breathing trial. In another embodiment, method 300 displays that the basis for ending the spontaneous breathing trial was a user entered command. In a further embodiment, method 300 displays that the basis for ending the spontaneous breathing trial was that at least one of the plurality of sensor measurements was outside of the desired range for the predetermined amount of time and/or the RSBI calculation was outside of the desired range for the predetermined amount of time. In an additional embodiment, method 300 further displays at least one of breath type, pressure support level, oxygen percentage of the gas mixture, PEEP, for the spontaneous breathing trial, and the remaining amount of time for the spontaneous breathing trial period.

Numerous other changes may be made which will readily suggest themselves to those skilled in the art and which are encompassed in the spirit of the disclosure and as defined in the appended claims. While various embodiments have been described for purposes of this disclosure, various changes and modifications may be made which are well within the scope of the present invention. Numerous other changes may be made which will readily suggest themselves to those skilled in the art and which are encompassed in the spirit of the disclosure and as defined in the appended claims.

1.-20. (canceled)

21. A method for managing a spontaneous breathing trial in a medical ventilator, comprising:
receiving a spontaneous breathing trial threshold for a rapid shallow breathing index (RSBI);
receiving a spontaneous breathing trial threshold for a tidal volume;
receiving a spontaneous breathing trial threshold for an exhalation volume;
initiating a spontaneous breathing trial for a patient being ventilated on a medical ventilator;
receiving data from a plurality of sensors, the data including a plurality of sensor measurements taken during the spontaneous breathing trial, wherein the plurality of sensor measurements include a measured flow rate, a measured tidal volume, and a measured exhalation volume;
determining a breath frequency based on the measured sensor measurements;
determining a RSBI based on the sensor measurements and the determined breath frequency;
displaying a graph of the determined RSBI, the measured tidal volume, and the measured exhalation volume versus time during the spontaneous breathing trial;
displaying the received breathing trial threshold for the tidal volume, the received spontaneous breathing trial threshold for the exhalation volume, and the received spontaneous breathing trial threshold for the RSBI on the graph; and
displaying a breach of the received breathing trial threshold for the tidal volume by the measured tidal volume, a breach of the received spontaneous breathing trial threshold for the exhalation volume by the measured exhalation volume, and a breach of the received spontaneous breathing trial threshold for the RSBI by the determined RSBI with a change in color on the graph.

22. The method of claim 21, further comprising:
ending the spontaneous breathing trial based on a first occurrence of the following:
a determination that the measured exhalation volume breached the received spontaneous breathing trial threshold for the exhalation volume for a predetermined amount of time,
a determination that the measured tidal volume breached the received spontaneous breathing trial threshold for the tidal volume for the predetermined amount of time,
a determination that the determined RSBI breached the received spontaneous breathing trial threshold for the RSBI for the predetermined amount of time,
a received user command, and
expiration of a spontaneous breathing trial period.

23. The method of claim 21, further comprising:
receiving a spontaneous breathing trial threshold for a blood oxygen saturation level;
wherein the plurality of sensor measurements include a measured blood oxygen saturation level;
wherein the step of displaying the graph further includes:
displaying the measured blood oxygen saturation level versus time for the spontaneous breathing trial on the graph;
displaying the received spontaneous breathing trial threshold for the blood oxygen saturation level on the graph; and
displaying a breach of the received the received spontaneous breathing trial threshold for the blood oxygen saturation level by the measured blood oxygen saturation level with a change in color on the graph.

24. The method of claim 21, further comprising:
receiving a spontaneous breathing trial threshold for a heart rate;
wherein the plurality of sensor measurements include a measured heart rate;
where the step of displaying the graph further includes:
displaying the measured heart rate versus time for the spontaneous breathing trial on the graph;
displaying the received spontaneous breathing trial threshold for the heart rate on the graph; and
displaying a breach of the received the received spontaneous breathing trial threshold for the heart rate by the measured heart rate with a change in color on the graph.

25. The method of claim 21, further comprising:
receiving a spontaneous breathing trial threshold for a carbon dioxide elimination level;
wherein the plurality of sensor measurements include a measured carbon dioxide elimination level;
wherein the step of displaying the graph further includes:
displaying the measured carbon dioxide elimination level versus time for the spontaneous breathing trial on the graph;
displaying the received spontaneous breathing trial threshold for the carbon dioxide elimination level on the graph; and
displaying a breach of the received the received spontaneous breathing trial threshold for the carbon dioxide elimination level by the measured carbon dioxide elimination level with a change in color on the graph.

26. The method of claim 21, further comprising:
receiving a spontaneous breathing trial threshold for a breathing work estimate;
determining a breathing work estimate based at least on the measured flow rate;
wherein the step of displaying the graph further includes:
displaying the determined breathing work estimate versus time for the spontaneous breathing trial on the graph;
displaying the received spontaneous breathing trial threshold for the breathing work estimate on the graph; and
displaying a breach of the received the received spontaneous breathing trial threshold for the breathing work estimate by the determined breathing work estimate with a change in color on the graph.

27. The method of claim 21, wherein the graph is displayed on a display that is separate and independent from a ventilator.

28. The method of claim 27, wherein the graph is displayed on the display that is separate and independent from a spontaneous breathing trial manager.

29. The method of claim 21, wherein the graph is displayed on a display that is separate and independent from a spontaneous breathing trial manager.

30. The method of claim 21, further comprising:
receiving a spontaneous breathing trial threshold for a breath frequency;
wherein the step of displaying the graph further includes:
displaying the determined breath frequency versus time for the spontaneous breathing trial on the graph;
displaying the received spontaneous breathing trial threshold for the breath frequency on the graph; and
displaying a breach of the received the received spontaneous breathing trial threshold for the breath frequency by the determined breath frequency with a change in color on the graph.

31. The method of claim 21, further comprising:
displaying a notification that the spontaneous breathing trial should be ended based on a first occurrence of the following:
a determination that the measured exhalation volume breached the received spontaneous breathing trial threshold for the exhalation volume for a predetermined amount of time,
a determination that the measured tidal volume breached the received spontaneous breathing trial threshold for the tidal volume for the predetermined amount of time,
a determination that the determined RSBI breached the received spontaneous breathing trial threshold for the RSBI for the predetermined amount of time,
a received user command, and
expiration of a spontaneous breathing trial period.

32. A medical ventilator system, comprising:
a processor;
a gas regulator controlled by the processor, the gas regulator adapted to regulate a flow of gas from a gas supply to a patient via a patient circuit;
a tidal volume sensor monitored by the processor, the tidal volume sensor is adapted to measure tidal volume of the patient;
a flow sensor monitored by the processor, the flow sensor is adapted to measure the flow of the gas to and from the patient;
an exhalation volume sensor monitored by the processor, the exhalation volume sensor is adapted to measure an exhalation volume of the patient;
a spontaneous breathing trial manager in communication with the processor the spontaneous breathing trial manager including:
a threshold monitor module, the threshold monitor module monitors at least one of the measured tidal volume, the measured exhalation volume, and a determined RSBI during a spontaneous breathing trail and compares the measured tidal volume, the measured exhalation volume, and the determined RSBI to a breathing trial threshold for the tidal volume, a spontaneous breathing trial threshold for the exhalation volume, and a spontaneous breathing trial threshold for the RSBI, and
a ventilation module, the ventilation module initiates and ends the spontaneous breathing trail by sending commands to the processor; and
a display module, the display module displays a software-generated graphic of a graph, wherein the graph includes:
a determined RSBI, a measured tidal volume, and a measured exhalation volume versus time for the spontaneous breathing trial, and
the spontaneous breathing trial threshold for the tidal volume, the spontaneous breathing trial threshold for the exhalation volume, and the spontaneous breathing trial threshold for the RSBI.

33. The medical ventilator system of claim 32, wherein the spontaneous breathing trial manager is a separate and an independent component from a ventilator.

34. The medical ventilator system of claim 33, wherein the display module is a separate and an independent component from a ventilator.

35. The medical ventilator system claim 32, wherein the display module is a separate and an independent component from a ventilator.

36. The medical ventilator system claim 32, wherein the graphic illustrates a breach of the received breathing trial threshold for the tidal volume by the measured tidal volume, a breach of the received spontaneous breathing trial threshold for the exhalation volume by the measured exhalation volume, and a breach of the received spontaneous breathing trial threshold for the RSBI by the determined RSBI with a change in color on the graph.

37. The medical ventilator system of claim 32, further comprising:
a blood oxygen saturation level sensor monitored by the processor, the a blood oxygen saturation level sensor is adapted to measure a blood oxygen saturation level of the patient;
wherein the graph further includes:
a measured blood oxygen saturation level versus time for the spontaneous breathing trial, and
the spontaneous breathing trial threshold for the blood oxygen saturation level.

38. The medical ventilator system of claim 32, wherein the processor generates the software-generated graphic of the graph.

39. The medical ventilator system of claim 32, wherein the spontaneous breath trial manager includes a spontaneous breathing trial manager processor, wherein the spontaneous breathing trial manager processor generates the software-generated graphic of the graph.

40. A computer-readable medium having computer-executable instructions for performing a method for managing a spontaneous breathing trial in a medical ventilator, comprising:
receiving a spontaneous breathing trial threshold for a rapid shallow breathing index (RSBI);
receiving a spontaneous breathing trial threshold for a tidal volume;
receiving a spontaneous breathing trial threshold for an exhalation volume;
repeatedly initiating a spontaneous breathing trial for a patient being ventilated on a medical ventilator;
repeatedly receiving data from a plurality of sensors, the data including a plurality of sensor measurements taken during the spontaneous breathing trial, wherein the plurality of sensor measurements include a measured flow rate, a measured tidal volume, and a measured exhalation volume;
repeatedly determining a breath frequency based on the measured sensor measurements;
repeatedly determining a RSBI based on the sensor measurements and the determined breath frequency;
repeatedly displaying a graph of the determined RSBI, the measured tidal volume, and the measured exhalation volume versus time during the spontaneous breathing trial;
repeatedly displaying the received breathing trial threshold for the tidal volume, the received spontaneous breathing trial threshold for the exhalation volume, and the received spontaneous breathing trial threshold for the RSBI on the graph; and
repeatedly displaying a breach of the received breathing trial threshold for the tidal volume by the measured tidal volume, a breach of the received spontaneous breathing trial threshold for the exhalation volume by the measured exhalation volume, and a breach of the received spontaneous breathing trial threshold for the RSBI by the determined RSBI with a change in color on the graph.
